(19) United States
(12) Patent Application Publication
(10) Pub. No.: US 2012/0145167 A1
(43) Pub. Date: Jun. 14, 2012

(54) Splinted therapeutic footwear

(76) Inventor: William Davis, Utica, MN (US)

(21) Appl. No.: 13/385,132

(22) Filed: Feb. 3, 2012

Related U.S. Application Data
(63) Continuation-in-part of application No. 11/904,561, filed on Sep. 27, 2007.

(51) Int. Cl. A61F 5/37

(52) U.S. Cl. 128/882

(57) ABSTRACT

A therapeutic fabric boot that warms the calf and foot, permits ambulation and provides for moisture absorption and selective exposure and ventilation of the covered extremity. An internal foam cradle restricts foot movement and reduces pressure at the heel and calf when lying in a supine position. A re-sizable, flat, hinged foam extension piece extends from the cradle and folds to protect the heel, sole and toes. Several fleece lined fabric cover panels selectively fasten about the foot and calf via hook and loop fasteners. An L-shaped splint mounts in a pocket space of the boot and offset calf and heel portions of the splint further distribute the weight of a wearer's leg and foot away from the wearer's heel and onto a resting support surface.

### RELATED APPLICATION DATA

This is a continuation-in-part application of pending application U.S. Ser. No. 11/904,561, filed on Sep. 27, 2007.

### BACKGROUND OF THE INVENTION

The present invention relates to medical footwear and, in particular, to an integrated splint and ventilated, fleece lined fabric boot containing a contoured foam cradle piece and a foam extension piece that extends between the fabric and fleece layers from the cradle piece into a toe panel piece and wherein when the boot is positioned by the user in a supine resting position the splint and foam cradle piece cooperate to minimize contact pressure between the protected foot and boot, stabilize the foot relative to the boot and a resting support, and reduce leg elevation.

A variety of leg and footwear appliances have been developed for patient therapy situations. Pre and post-operative footwear are worn to prevent vasoconstriction and promote vasodilatation to maintain blood circulation and thereby prevent clotting. That is, by keeping the legs and feet warm, the blood vessels don't constrict and healthy blood flow is maintained. The footwear also physically protects and warms the legs and feet with minimal skin trauma (e.g. ulcerations, cracking and/or abrasions). Fleece-lined, hook and loop fastened boots of the foregoing type manufactured by applicant are disclosed in U.S. Pat. No. Des. 326,556 and 6,866,043.

The present improved footwear was developed to provide a splinted therapeutic fabric boot that warms the extremity, permits ambulation and reduces supine contact pressure at the foot. The improved boot contains a foam cradle piece having a flat posterior surface and an anterior surface contoured to mate with the calf and support and elevate the heel with minimal heel contact pressure with the boot.

A plantar foam sole piece is hinged to extend from the foam cradle piece and can be cut to length to tailor the boot size to best fit the user. One end of the foam sole piece is fastened to a flat posterior surface of the foam calf support, extends beneath the sole and toes and an opposite end folds to over the ends of the toes onto an anterior surface of the foot. The boot interior is lined and faced with insulation and fleece. Numerous hook and loop fastener pieces are fitted to exterior cloth surfaces and attach to straps and/or fabric cover pieces upon wrapping the straps and cover pieces about a supported calf and foot.

In combination with a malleable splint fitted to an external pocket underlying the plantar foam extension piece and sole and fastened with straps to the external boot shell, the foam support and splint prevent leg rotation, reduce leg elevation and collectively minimize contact pressure between the heel and boot when the boot and leg are supported in a supine posture.

### SUMMARY OF THE INVENTION

It is accordingly a primary object of the invention to provide thermally insulated medical footwear to stimulate blood circulation.

It is a further object of the invention to provide pre and post-operative footwear comprising a lined, thermally insulated, moisture wicking fabric boot having a number of hook and loop fasteners fitted to boot panel pieces and associated straps to collectively wrap and fasten to configure the boot about the leg and foot.

It is a further object of the invention to provide a fleece and laminated, porous moisture wicking fabric lined boot with a foam insert to define a flat posterior surface and a contoured interior surface that supports the calf and heel to prevent rotation and minimize heel support pressure when resting in a supine posture.

It is a further object of the invention to provide a foam support having a conformal contoured anterior surface that supports the calf and elevates the heel to reduce pressure at the heel when resting in a supine posture.

It is a further object of the invention to provide a foam support having a seamless, hinged plantar extension piece that can be cut to size and that wraps to encase the heel sole and toes.

It is a further object of the invention to provide a boot with fleece and/or porous, moisture wicking liner pieces that overlie and contain a foam support(s).

It is a further object of the invention to provide a boot with panel pieces that permit selective exposure of portions of the foot and/or calf to inspection and ventilation without disruption to other panels.

It is a further object of the invention to provide an accessory wedge piece that mounts to the boot to support the foot against rotation.

It is a further object of the invention to provide straps that wrap around the metatarsal heads and attach to the boot to support the toes and prevent “foot drop” and accommodate swelling or bulky bandaging or dressings.

It is a further object of the invention to provide a resilient, malleable splint fitted to underlie the plantar foam extension piece and sole and fastened to external boot shell to cooperate with the foam cradle and plantar extension pieces to prevent leg rotation, reduce leg elevation and collectively minimize contact pressure between the heel and boot when the boot and leg are supported in a supine posture.

The foregoing objects, advantages and distinctions of the invention are obtained in a presently preferred fabric boot of the invention that is lined with thermal insulation, fleece and/or porous, laminated fabric. The laminated fabric is constructed and located to enhance the wicking of moisture and ventilation of the foot. Several tabs of hook and loop fastener materials are arrayed about several fabric panel pieces that mate with other associated panel pieces and straps. The straps and panel pieces align to define and selectively control the fitting of the boot to the foot. The panel pieces are shaped and located to permit selective exposure of portions of the foot without disrupting adjoining panel pieces that cover other portions of the foot to permit inspection and ventilation.

A contoured foam calf and heel support cradle mounts within the boot. Ventilation apertures can be formed in the foam support. A posterior surface of the calf and heel support cradle exhibits a flat posterior surface that prevents leg rotation. A longitudinal interior or anterior surface of the calf and heel support cradle is contoured to conform to the calf, elevate the heel and distribute leg weight to prevent pressure points, especially at the heel. The calf and heel cradle support is contained in a pocket space between the external fabric cover and an internal liner.

A foam plantar extension piece is fastened and hinged to the calf/heel support at a recessed pocket. A closed cell foam portion of the extension piece is covered with an open cell foam piece and fleece and/or fabric materials. The plantar extension piece extends between the fabric and fleece layers of foot panels into a toe panel. Lateral flaps or wings of the foot panels project from the sides of the covered extension piece to overlap the foot. The wings are secured to permit selective exposure and/or ventilation of the foot without disrupting other, separately fastened panel pieces. The plantar extension piece underlies the heel, sole and toes and folds at the toe panel to protect the distal ends of the toes. The wing pieces of the foot panels and toe panel can be trimmed at stitching lines displaced to accommodate different foot sizes to re-size and tailor fit the boot.

Hook and loop fasteners are secured to external and internal panel surfaces of the panels and are aligned to overlap and secure the boot to the foot. An accessory wedge piece fastens to the external cover to prevent leg and/or foot rotation. Other accessory and extension straps mount to the external fabric layer of the panels to prevent foot drop and accommodate swelling and/or bulky bandaging or dressings.

A resilient, malleable, generally L-shaped splint is fitted to a pocket at the sole of the boot to underlie the plantar foam extension piece and wearer's sole. A foam layer having hook/loop fasteners covers a portion of the splint. A fabric sleeve contains a portion of the splint and the sleeve and splint to the pocket via the hook/loop fasteners. Hook/loop straps threaded to the splint fasten to calf panels and boot shell. The splint cooperates with the foam cradle and plantar extension pieces to prevent leg rotation, reduce leg elevation and minimize contact pressure between the boot and heel when the boot and wearer's leg are supported in a supine posture.

Still other objects, advantages, distinctions and constructions of the invention will become more apparent from the following description with respect to the appended drawings. Similar components and assemblies are referred to in the various drawings with similar alphanumeric reference characters. The description should not be literally construed in limitation of the invention. Rather, the invention should be interpreted within the broad scope of the further appended claims.

### BRIEF DESCRIPTION OF THE DRAWINGS

FIG. 1 is a right side perspective drawing of the boot shown in an upright position with the boot wrapped to a closed condition and a foot drop strap mounted to the cover.

FIG. 2 is a left side perspective drawing of the boot with the boot wrapped to a closed condition and a foot drop strap mounted to the cover.

FIG. 3 is a rear perspective drawing of the boot with the boot wrapped to a closed condition and a foot drop strap mounted to the cover.

FIG. 4 is a bottom perspective drawing of the boot with the boot wrapped to a closed condition and a foot drop strap mounted to the cover.

FIG. 5 is a front perspective drawing of the boot with the boot wrapped to a closed condition and a foot drop strap mounted to the cover.

FIG. 6 is a front perspective drawing showing the calf and plantar, heel/sole/toe extension pieces and lateral overlapping wing panel pieces folded open and detached from the calf support panels.

FIG. 7 is a front perspective drawing showing an alternative construction of the boot liner wherein a porous, moisture wicking liner covers the calf and heel support surfaces of the boot.

FIG. 8 is a rear perspective drawing showing the external surface of the boot folded open.

FIG. 9 is a front perspective drawing showing the foam calf cradle support and foam plantar heel/sole/toe extension piece removed from the boot.

FIG. 10 is a longitudinal cross section drawing of the calf support cradle depicting the flat outer and contoured inner surfaces.

FIG. 11 is a front perspective drawing showing the heel/sole/toe extension piece and the overlapping wing panels folded open.

FIG. 12 is a front perspective drawing showing the toe portion of the plantar extension piece folded closed and the adjacent overlapping wing panels folded open.

FIG. 13 is a perspective drawing showing a strap that mounts to the boot to prevent foot drop.

FIG. 14 is a perspective drawing showing an accessory wedge support piece that mounts to prevent foot rotation.

FIG. 15 is a perspective drawing showing the foot drop strap and accessory wedge as they appear when mounted to the boot and the foot of a supine user.

FIG. 16 is a pressure map drawing showing the foot pressure of a prior art boot having an average pressure distribution of 16 mmHg and several peak pressure points on the order of 62 mmHg.

FIG. 17 is a pressure map drawing showing the foot pressure of the improved boot of the invention with a reduced, evenly distributed pressure profile on the order of 14 mmHg and a minor peak pressure of 28 mmHg.

FIG. 18 shows a perspective view of a resilient, malleable splint partially mounted to an external pocket that extends beneath the sole of the boot and with associated strap fasteners released.

FIG. 19 shows a longitudinal cross section view of the boot and splint resting in a supine position with the splint mounted to the boot and without the strap fasteners and wherein a wearer's calf and foot are shown in dashed line.

FIG. 20 shows a perspective view of the resilient, malleable splint of FIGS. 18 and 19 uncovered and removed from the boot.

Similar structure throughout the drawings is referred to with the same alphanumeric reference numerals and/or characters.

### DESCRIPTION OF THE PREFERRED EMBODIMENT

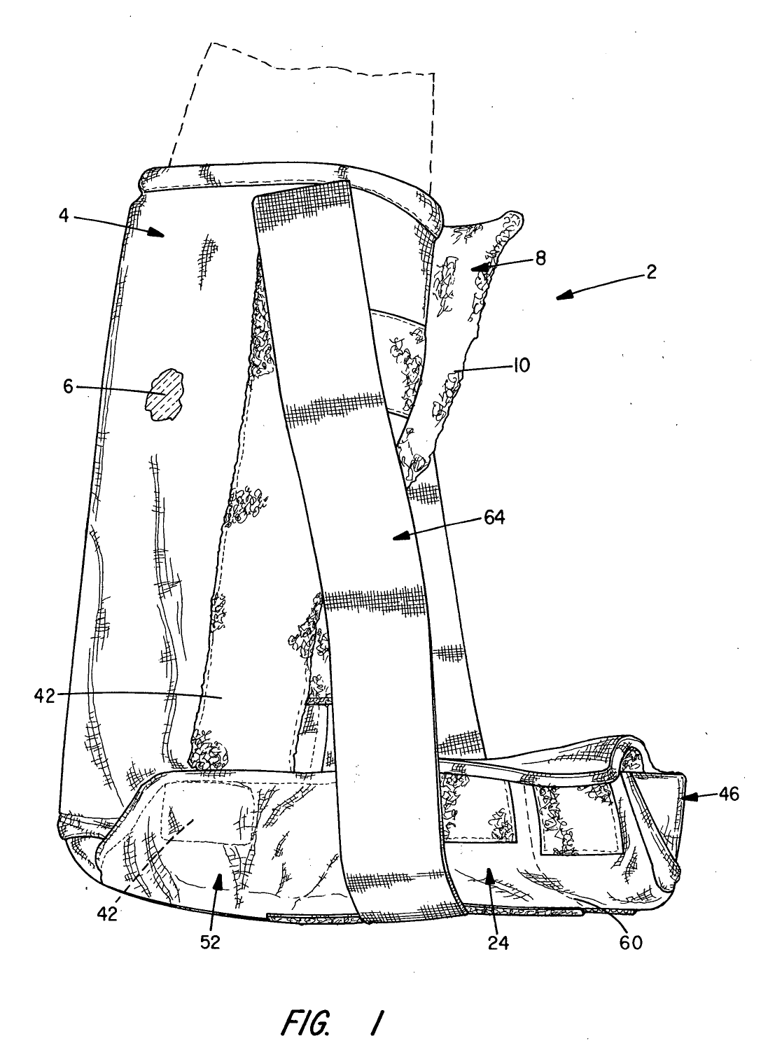

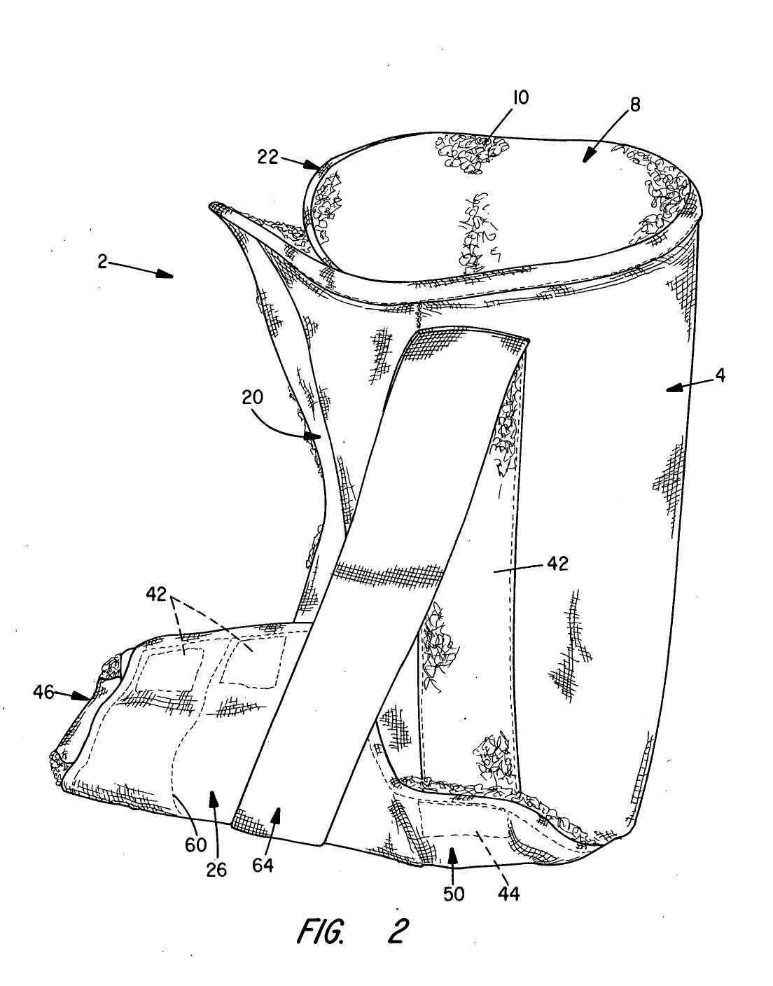

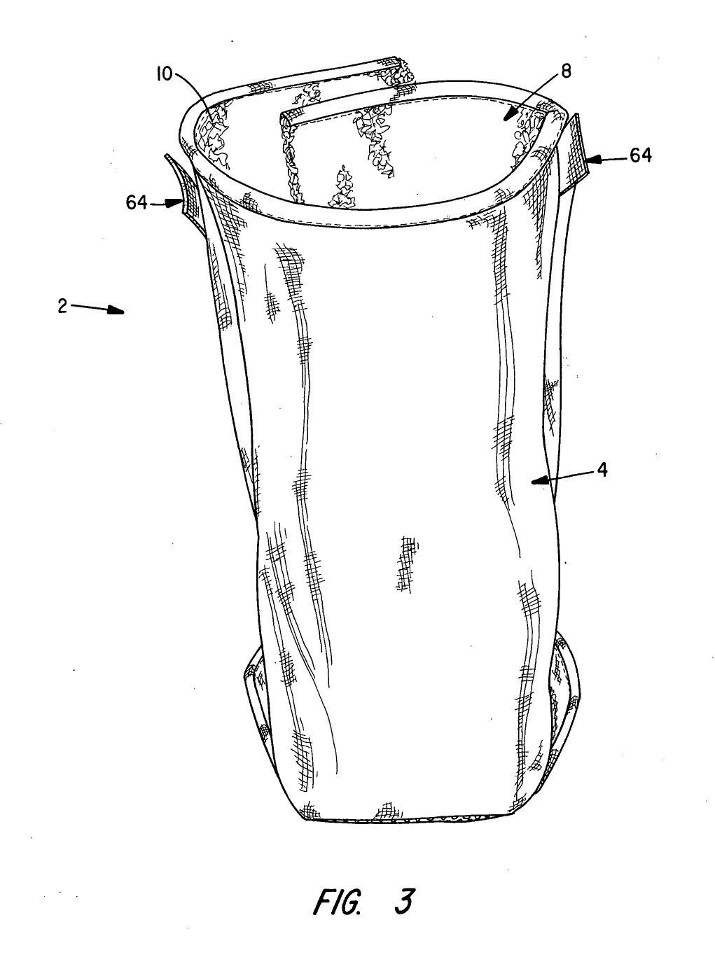

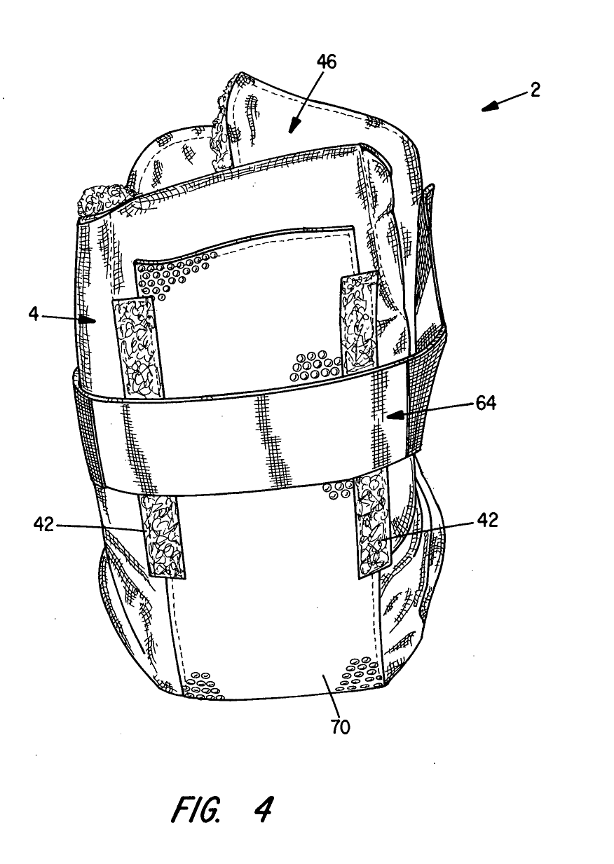

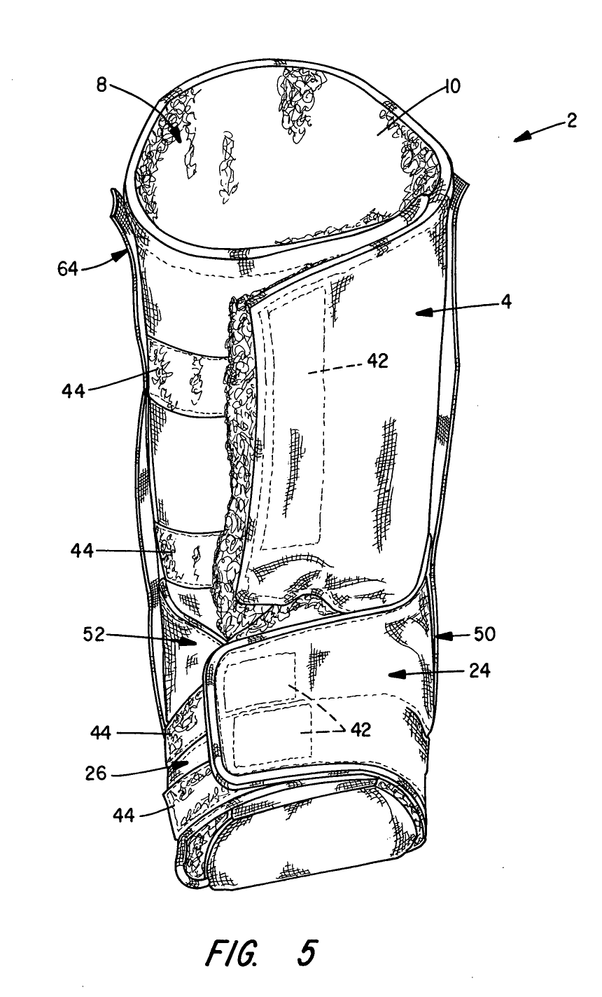

Referring to FIGS. 1-5 several perspective views are shown to external surfaces of the improved therapeutic boot 2 of the invention. The boot 2 is constructed of an air permeable, fabric cover 4. The cover 4 is presently sewn from a durable velour cloth. Other materials such as a heavyweight cotton fabric, CORDURA® or other fabrics or fabric combinations might also be used.

A thermal insulation material 6 (shown in cutaway) is fitted between the cover 4 and a separate interior lining 8. The insulation material 6 can comprise THINSULATE® or any of a variety of other suitable thermal insulation materials. The insulation material 6 provides a thermal barrier to maintain the temperature of the calf and foot to promote dilation of the blood vessels and blood flow through the covered extremity.

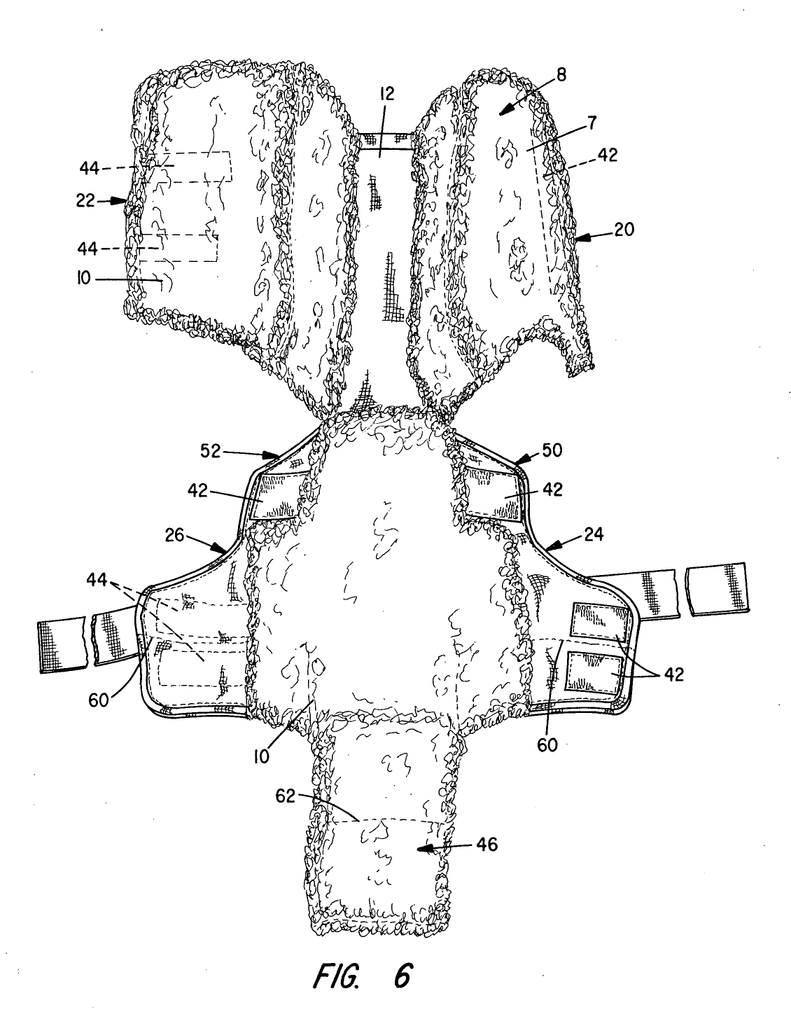

With additional attention to FIGS. 6, 7 and 9, the lining 8 is constructed and organized to provide a soft, smooth/comfortable surface to contact the skin without any abrading seams or surface portions that can cause ulcerations or skin trauma. The lining 8 is also constructed to provide a soft interface with the calf and foot to wick moisture away and ventilate the covered calf and foot. Seams between sections of fleece 10 and/or fabric, moisture wicking materials 12 are located and configured to minimize contact with the skin. Seams can be sewn or adhesively fixed.

Seams in the region of the heel are especially avoided or hidden in the boot 2. Presently, an adhesively bound seam between calf and extension liner pieces at the heel is located in or immediately adjacent a recess 32 of a foam support cradle 28. The recess 32 shelters overlapping or butted, adhesively bound liner pieces. The recess 32 also shelters the proximal end of a foam, plantar extension piece 30 that extend from the support cradle 28. regions of sustained contact between the skin and the lining 8.

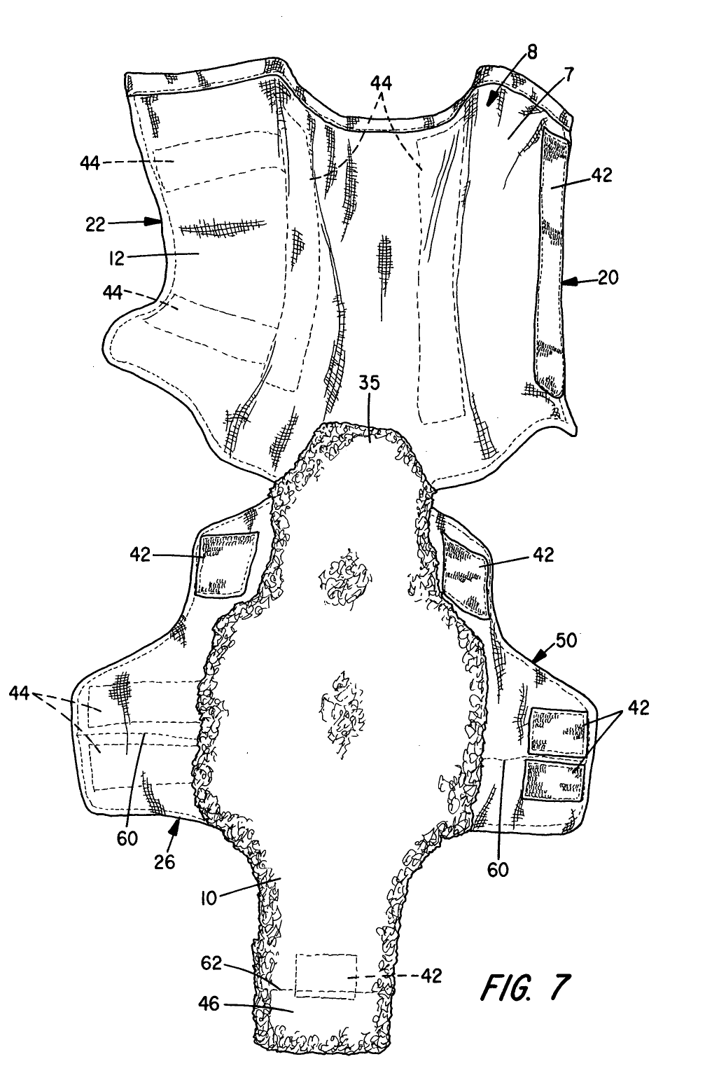

For the boot 2 of FIG. 6, the fleece material 10 comprises the entire liner 8 and is located to surround the calf and foot. As with the boot 2 of FIG. 7, the insulation 6 is generally included beneath all regions of the lining 8 to surround the entire calf and foot, although could be selectively positioned about the boot 2. Collectively, the insulation 6 and fleece material 10 traps warm air and wicks away moisture.

The interior lining 8 of the boot 2 of FIG. 7 otherwise is constructed of several sections of fleece material 10 and/or moisture wicking materials 12. The moisture wicking material 12 used in the boot of FIG. 7 presently comprises a material having a porous, center substrate piece and to the opposite surfaces of which micro-fleece materials are laminated. The side facing the interior of the boot 2 is covered with an open-weave and/or porous polyester facing piece (e.g. such as used in sports clothing). The facing piece is smooth and apertures in the material ventilate the material and foot against and any absorbed moisture. A variety of other soft, moisture absorbent, air permeable open weave or porous materials can be used.

The wicking material 12 is located to absorb moisture and ventilate the foot. Moisture typically develops at regions of sustained contact between the skin and the lining 8. The moisture wicking material 12 is preferably located at the primary points of contact with the calf and foot, for example, at the posterior surfaces of the calf, at the heel, sole and around the foot.

The boot 2 of FIG. 7 only surrounds the calf with the foregoing laminated moisture wicking materials 12. Fleece 12 otherwise covers the foot and a layer of insulation 6 is provided below both materials 10 and 12. While FIGS. 6 and 7 depict two presently preferred configurations of the liner 8, the locations of the fleece 10 and moisture wicking material 12 can be varied as desired.

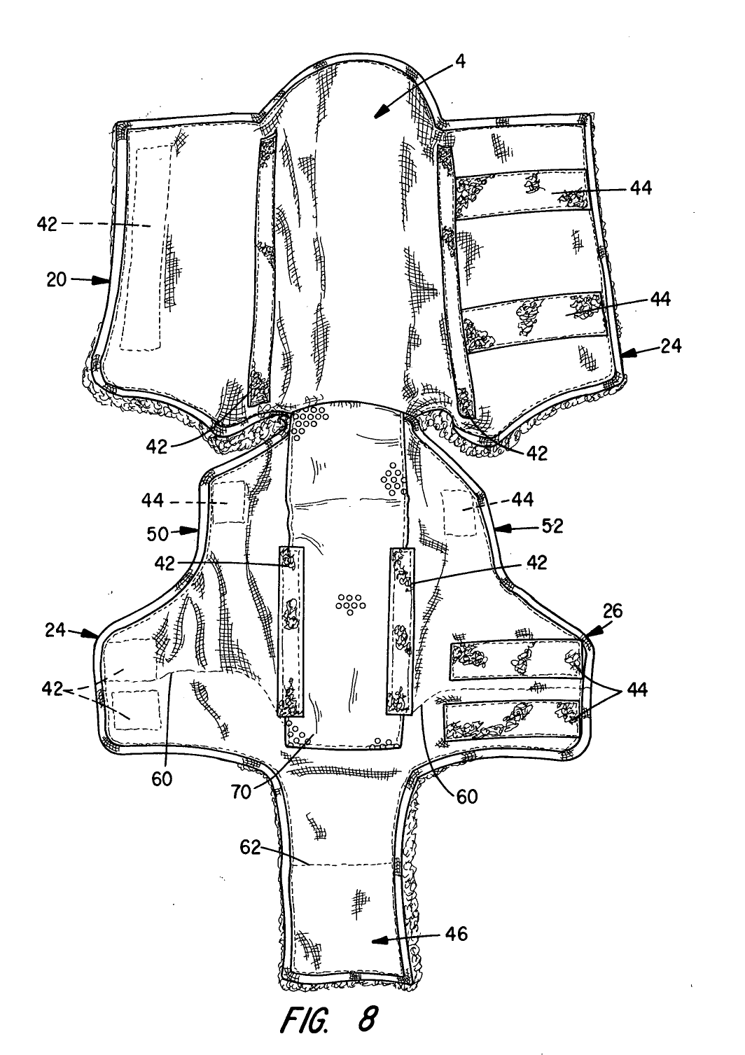

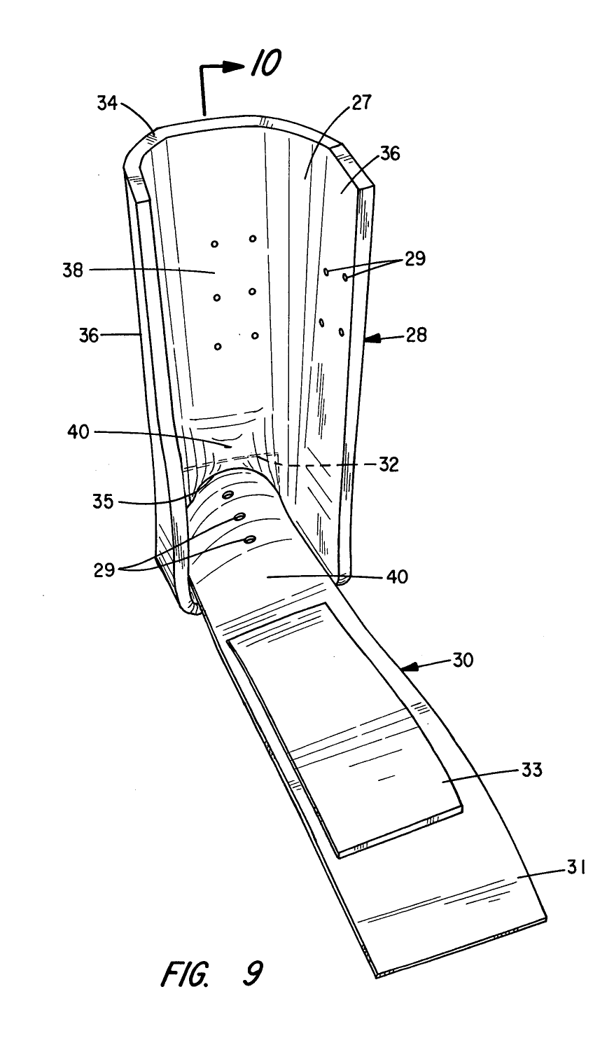

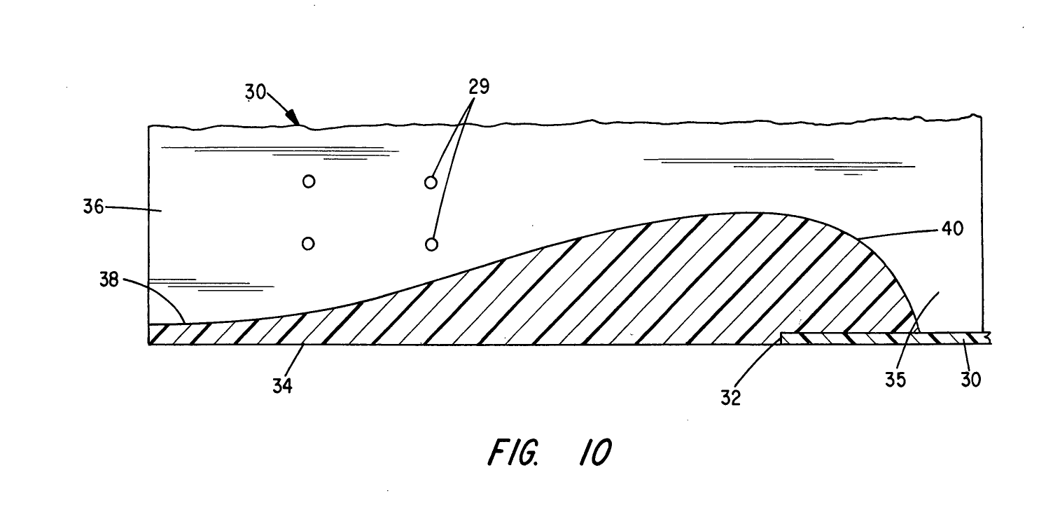

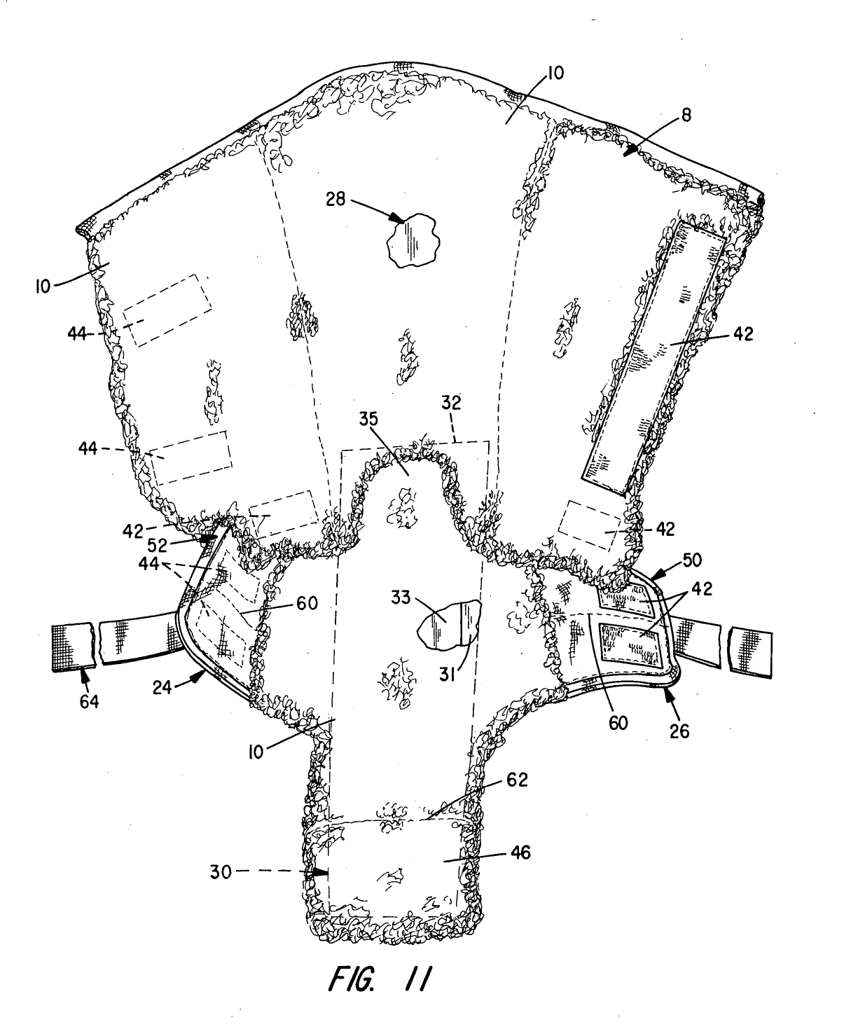

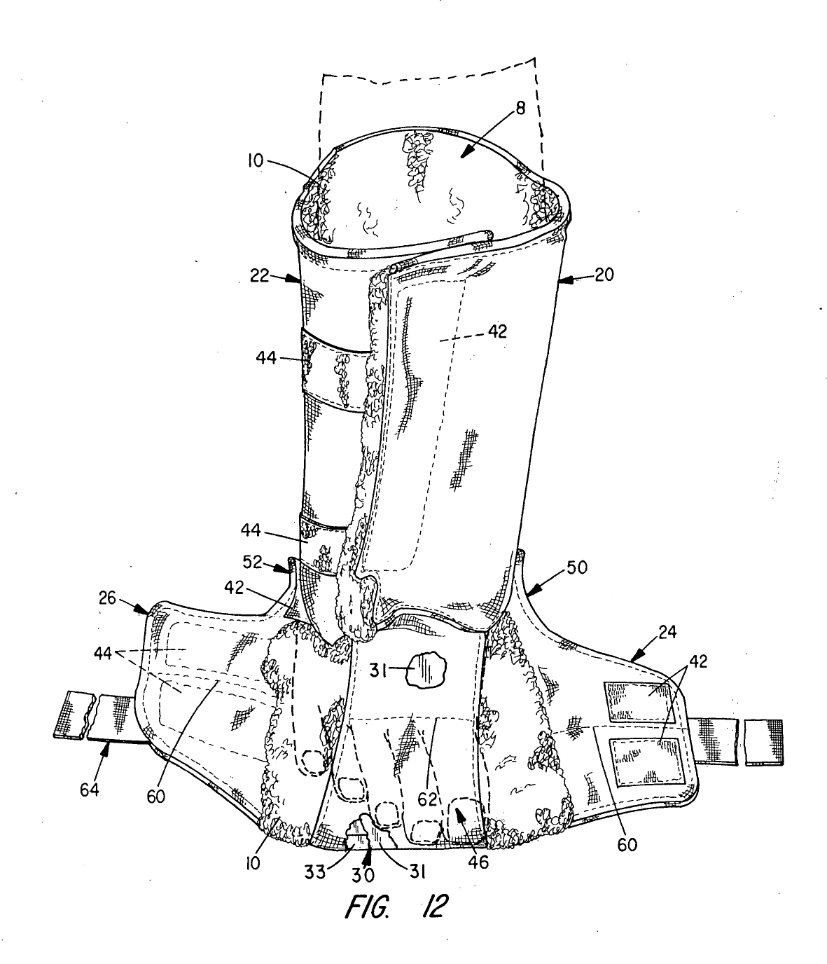

With additional attention to FIGS. 6 through 9, details are shown to the construction of the boot 2 and the relative arrangement of several fabric panels that are or can be lined with the fleece 10 and/or moisture wicking material 12. FIGS. 6 through 8 depict the interior and exterior construction of the boot 2 with the panels folded open. FIGS. 9 and 10 depict the foam support cradle 28 that mounts inside the boot 2 beneath the liner 8. FIGS. 11 and 12 depict the several boot panels as they are sequentially folded and fitted to the calf and foot to ultimately form the wrapped configurations shown at FIGS. 1 through 5.

Apparent from FIGS. 6 through 8 are respective internal and external folded open views of the boot 2. The boot 2 opens full-length along its front or anterior surface at right and left, fleece covered calf panels 20 and 22 and right and left foot panels 24 and 26.

The calf panels 20 and 22 overly and contain the foam support cradle 28 shown in detail at FIGS. 9 and 10. The cradle 28 is preferably constructed of a relatively dense elastic material that flexes or compresses slightly to provide a resilient interface with the calf. The cradle 28 can for example be constructed of a variety of materials including elastomers, polyurethane foam, and/or other open and/or closed cell foams or combinations thereof. The durometer and resilience of the material preferably compresses slightly to support the calf and foot without undue compression, yet springs back to shape upon relieving any pressure, particularly at the anterior surface of the calf.

The cradle 28 provides a longitudinal channel 27 that receives the calf The cradle 28 mounts in a pocket or space formed beneath and between the calf panels 20 and 22. The cradle 28 can provide through apertures 29 to aerate the calf The foot panels 24 and 26 overly and contain a separate flat, foam plantar extension piece 30. A base portion 31 of the extension piece 30 is constructed of a closed cell foam, approximately ⅛-inch thick. A proximal end is bonded to a recess 32 (shown in dashed line) formed into the flat posterior surface of the support cradle 28 adjacent an inverted U-shaped cutout 35 adjacent the peripheral edge of the anterior surface of the cradle 28. The cutout 35 is formed to shelter the suspended heel as described in more detail below. Peripheral edges of the cutout 35 arcuately depend to contact the extension piece 30, reference FIG. 10. Any seam between the cradle 28 and extension piece 30 is thereby removed and/or shielded by the recess 32 and cutout 35 from contact with the heel.

The plantar extension piece 30 extends from the region adjacent the heel and wraps over the sole and toes. An interior surface of the extension piece 30 is typically covered with a separate panel of open celled foam 33 approximately ½-inch thick. The plantar extension piece 30 and portions of the overlapping panels 24 and 26 are also fabricated to be sheared to size when the boot 2 is first fit to a user and the details to which fitting are discussed below. The extension piece 30 includes vent apertures 29 in the region of the heel.

With attention also to FIGS. 9 and 10, the support cradle 28, when viewed from a longitudinal end, generally exhibits a U-shape. A posterior surface 34 of the cradle 28 is constructed to be flat and from which side walls 36 project and extend along the longitudinal sides of the calf. The flat surface 34 stabilizes the cradle 28 and calf against rotation when the user rests in a supine position and the boot 2 is supported on a bed.

The anterior surface 38 of the cradle along the channel 27 is contoured and exhibits a compound arcuate shape to support the calf, reference FIG. 10. A raised surface portion 40 in the region of the Achilles tendon is shaped to elevate and support the heel in the cutout region 35 with minimal pressure and contact with the boot and away from any support structure, such as a bed, foot stool etc.

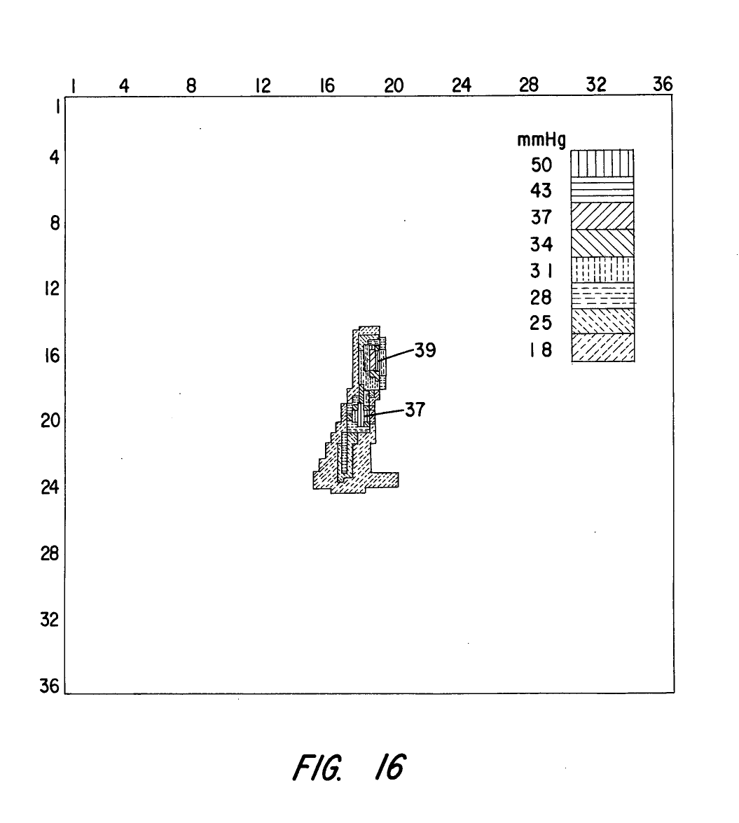

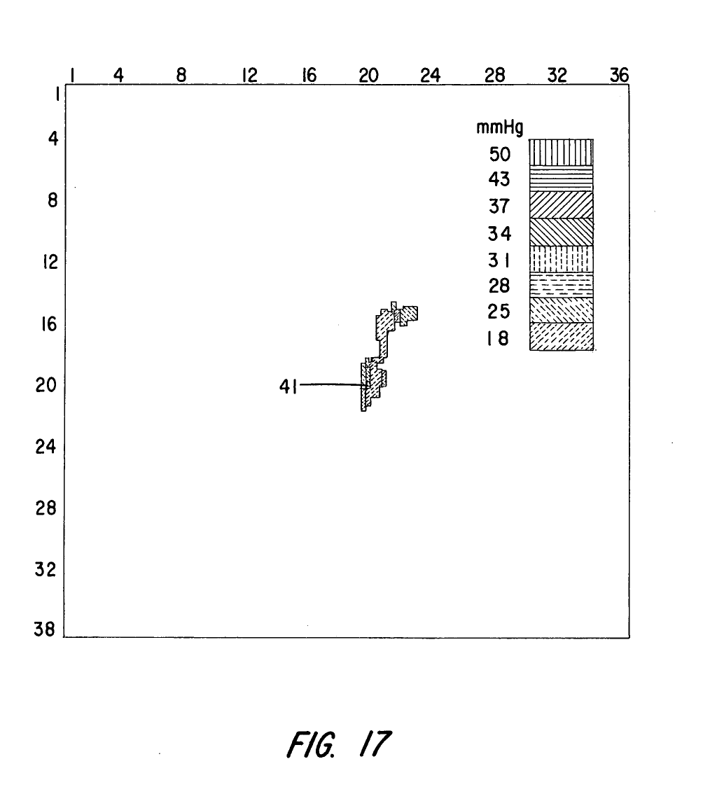

The contoured surfaces 38 and 40 are particularly formed to distribute and equalize support pressures on the calf and prevent pressure points that might induce skin ulceration or abrasion. FIG. 16 depicts a pressure map to a prior boot of applicant that exhibited an average pressure distribution of 16 mmHg and two relatively significant pressure points 37 and 39 on the order of 62 mmHg. FIG. 17 in contrast depicts a pressure map to the improved boot 2 of the subject invention which uses the cradle 28 and plantar extension piece 30 and wherefrom a reduction of pressure points and even distribution of pressure on the order of 14 mmHg average and a minor peak pressure point 41 of 28 mmHg is apparent. The latter pressures are well below the nominal pressure of 32 mmHg where capillary closure and reduced blood perfusion and flow is observed with consequent risks of skin ulceration

Returning attention to FIGS. 6 through 8 and when folded closed, the calf panels 20, 22 and foot panels 24, 26 respectively wrap and overlap each other to form the boot 2 shown at FIGS. 1-5. The overlapped panels 20-26 are fastened together with mating strips of hook 42 and loop 44 type fastener material aligned along the peripheral edges of the panels 20-26. Hook and loop fasteners 42 and 44 are preferred although other fastenings might be adapted to the boot 2.

In the region of the foot, several parallel strips of hook fastener material 42 are sewn to extend in displaced, transverse relation along the edge of the panel 24 to overlap and mate with several transverse strips of loop fastener material 44 located along the edge of the panel 26. The overlapped panels 24 and 26 cover a toe panel 46 defined by a distal end of the extension panel 30 that folds to cover the toes.

The calf panel 20 separately contains spaced, transverse strips of loop material 44 along the edge of the panel 22 that overlap and mate with a longitudinal strip of hook fastener material 44 that extends along the longitudinal edge of the panel 20. When the panels 20 and 22 are wrapped around the calf and overlapped in the manner of FIG. 12, the strips of fasteners 42 and 44 bind the upper portion of the boot 2 to the calf at a desired, adjustable pressure.

Also apparent from FIGS. 6 through 8 are right and left heel panels 50 and 52. The panels 50 and 52 include tabs of hook material 42 that mate with the lower end of longitudinal strips of loop material that longitudinally extend along the approximate center of the panels 20 and 22. The heel panels 50 and 52 hinge the foot panels 24 and 26 to the calf panels 20 and 22. The heel panels 50 and 52 also advantageously facilitate a selective exposure of the heel during treatment. That is, the heel panels 50 and 52 can be detached independent of the foot and calf panels 20-26, either for inspection or for ventilation without disrupting the foot panels 20-26 to expose the heel area. This additional flexibility is very desirable to facilitate healing and ventilate and aerate the lining 8 to evaporate moisture. FIGS. 11 and 12 depict the hinge panels 50 and 52 secured to the cradle panels 20 and 22.

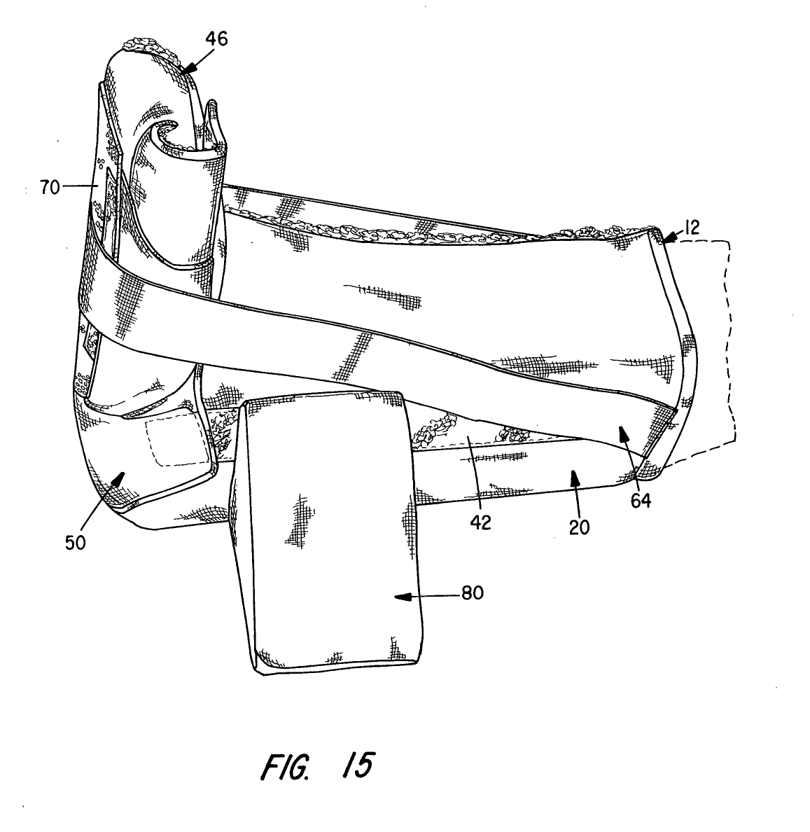

The boot 2 can also be re-sized to fit users with smaller feet. In this regard and from FIGS. 6 through 8, a number of seams of stitching 60 and 62 are respectively provided at the foot panels 24 and 26 and the toe panel 46. The stitching 60 and 62 permit a care giver to size the boot 2 to best fit the user. For patients with smaller feet, the distal portions of the toe and foot panels 46 and 24, 26 can be cut away or trimmed from the boot 2 to accommodate the smaller foot. The stitching 60 and 62 also prevents fraying at the remaining associated panel portions.

Where the flat posterior cradle surface 34 prevents foot rotation, a strap 64 shown at FIG. 12 is provided that can be secured to the longitudinal lengths of loop fastener material 42 at the cradle panels 24 and 26 and wrapped below the foot to prevent “foot drop”, reference FIGS. 1-5 and 15. The interior surface of the strap 64 is covered with several tabs of hook material 44. The strap 64 attaches beneath the foot panels 24 and 26 to strips of looped fastener 42 that longitudinally extend along the sides of a traction pad 70 that is formed from non-slip material and sewn to the surface of the cover 4 in the region of the sole.

The pad 70 facilitates safe ambulation over smooth tile or wood floors. The pad 70 can be constructed of a variety of non-slip materials. The pad 70 is shaped to essentially align with and underlie the plantar extension piece 30. Upon wrapping the panels 20-26 and 46 over the foot, the strap 64 overlaps mating loop material and establishes the angle of the foot to the calf.

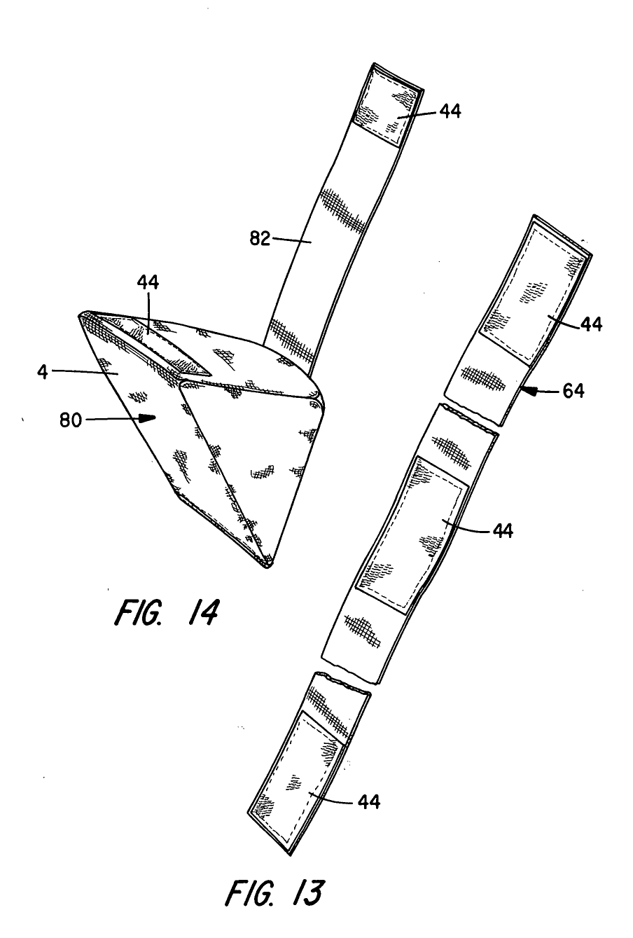

The foot can be further supported with the benefit of an accessory wedge-shaped support 80 shown at FIG. 13. The support 80 also prevents foot rotation. The support 80 is constructed of a right triangular piece of foam that is covered with the cover material 4. A tab of hook material 44 is secured to at least one of the exposed orthogonal surfaces and can fasten to the strips of loop material 42 that extend along the sidewalls of the calf panels 20 and 22.

A strap piece 82 and fastener tab 44 extend from the support 80 and can be trained beneath the calf panels 20 and 22 and fasten to strips of fastener 42 such as in the fashion shown at FIG. 14. The support 80 might also be fitted to axially align with the pad 70 and heel.

For users with relatively large feet, swelling or having bulky bandaging or dressings that prevent the foot panels 24 and 26 from overlapping, an extension strap of suitable length and constructed similar to strap 64 can also be secured between the foot panels 24 and 26.

From the foregoing, it is to be appreciated the boot 2 advantageously protects and thermally insulates the foot and lower leg. The construction of the boot 2 provides numerous advantages and improvements over known therapeutic footwear. The subject footwear particularly protects the heel and foot, permits ambulation, warms and ventilates the foot and calf.

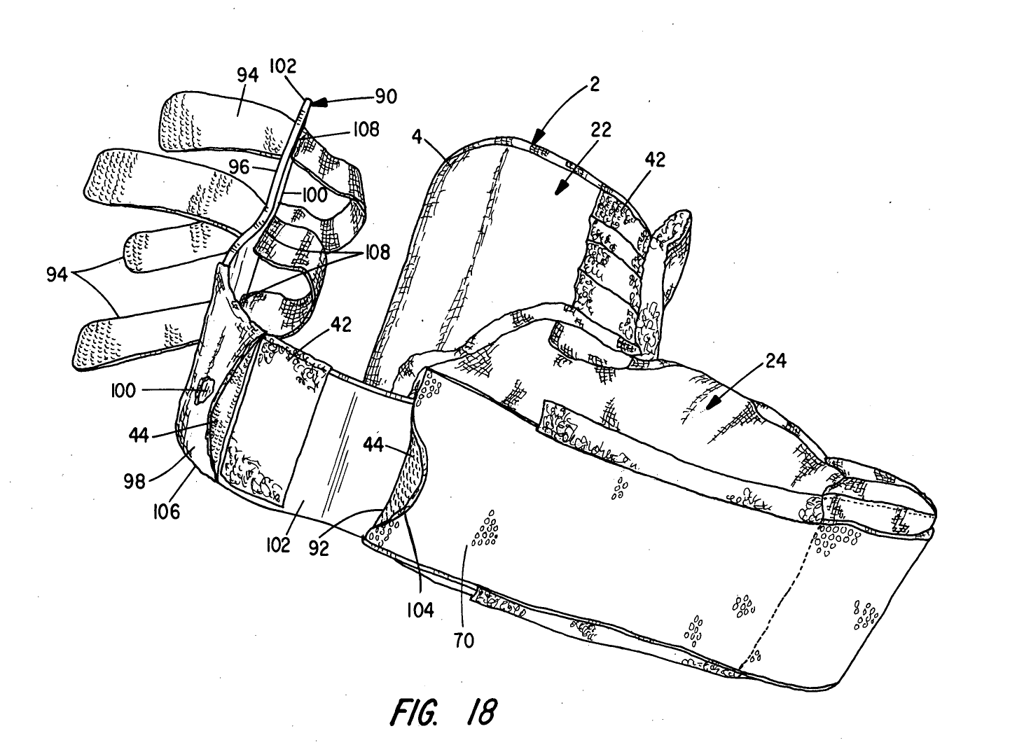

FIGS. 18 through 20 further depict a resilient, malleable splint 90 that mounts to the boot 2. The splint 90 mounts in a pocket space 92 formed between the traction pad 70 and outer fabric 4 at the sole of the boot 2. The splint 90 slides into the pocket space 92 and extends to a region beneath the toes. Several straps 94 are threaded through apertures 108 at the splint 90. The straps 94 fasten to strips of loop fastener material 42 that longitudinally extend along the sides of the calf panels 20 and 22.

The splint exhibits a generally L-shape. The splint 90 is bent to define a flat calf portion 96, a bent arcuate heel portion 98 and a flat sole portion 99. The calf portion 96 is elevated above the heel portion 98 in a range of approximately ⅜ to ¾-inch. When the heel portion 98 is positioned on a resting support 110 (e.g. a mattress, foot rest etc.), the heel portion 98 engages the resting support 110 and tends to slightly depress into the cushioned resting support 110 as the calf portion 96 distributes the weight of a wearer's leg and foot over the resting support 110. A wearer's calf thereby tends to lie flat on the resting support 110 without any bending at the knee which can be tiresome over prolonged periods. The depression of the heel portion 98 also prevents rotation of the boot 2 and the wearer's foot. The rigidity and secondary support of the splint 90 also prevents flexion of the flexible sidewalls 36 of the cradle 28 to prevent and minimize contact between the wearer's heel and the cradle 28 and interior surfaces of the boot 2.

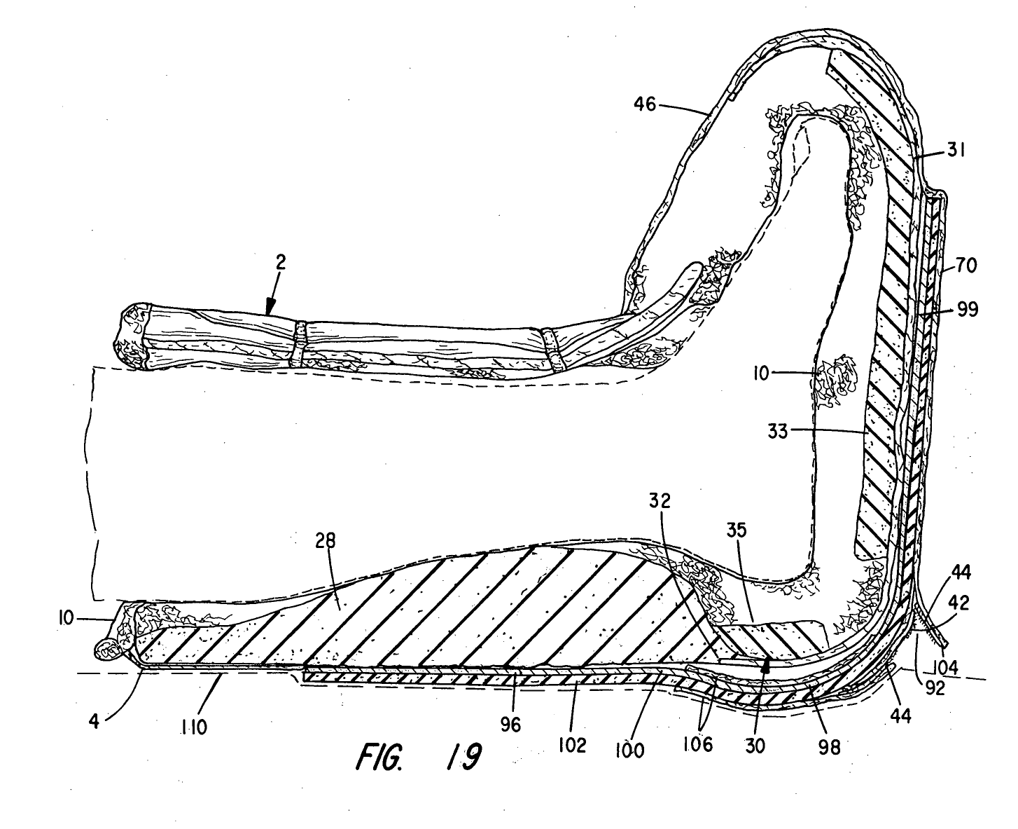

As noted at FIG. 19, the boot 2 is essentially displaced above the mattress 110 and the resting pressure on the boot 2 is substantially born by the leg, splint 90 and the relatively thick cradle 28. Contact pressure between the wearer's heel and the interior surfaces of the boot 2 is reduced versus using the boot 2 alone to support the wear's leg and foot. The weight of the wearer's leg and foot is distributed over the width of the splint 90 and the weight of the foot is transferred via the splint 90 to the leg and resting support 110.

The offset and elevated displacement of the flat horizontal calf portion 96 above the curvature of the flat heel portion 98 supplements the primary leg support provided by the foam cradle 28. The elevation and curvature of the heel portion 98 is formed to cooperate with the contours of the foam cradle 28 and cushioning of the foam plantar extension piece 30. As noted above, the wearer's heel is elevated in the boot 2 via contact of the calf with the cradle 28 to primarily minimize contact between the heel and the interior surfaces of the boot 2.

As noted above, the splint 90 stabilizes the boot 2 and thereby tends to fix the relative alignment of the foot to the calf in lieu of using the strap 64. The mating of the flat posterior surface of the cradle 28 to a flat anterior surface of the calf portion 96 and the depression of the heel portion 98 into the resting support 110 as the flat calf portion 96 rests on the flat resting support 110 stabilizes the boot 2 and foot against rotation at the resting support 110 (e.g. mattress) in lieu of using the wedge 80.

The support between the splint 90 and resting support 110 also acts to align the leg and calf more parallel to the mattress 110 versus if the splint 90 is not used. The weight of the leg and foot is thus substantially born by the mattress 110. The wearer's heel, in turn, is elevated to a parallel plane above the mattress 110 via the contour 40 and cutout space 35 of the cradle 28. The splint 90 along with the foam cradle and plantar extension pieces 28 and 30 separately and resiliently protects the elevated heel and foot encased in the boot 2.

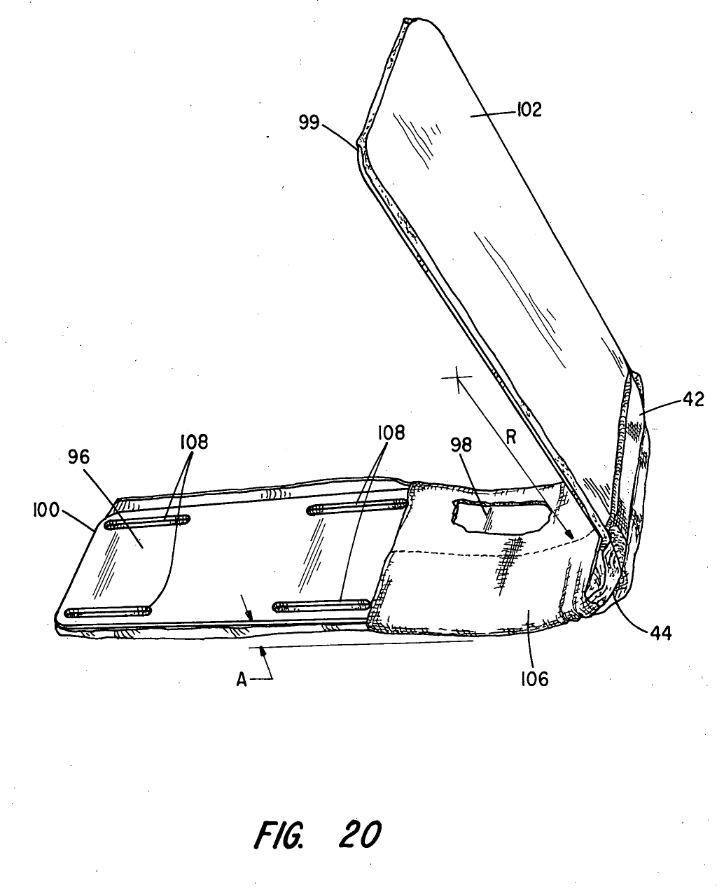

With attention to FIGS. 19 and 20, the splint 90 comprises an elongated metal member 100 that is covered on at least an exterior surface with a layer of closed cell foam 102. The internal surface of the splint can also be covered. The member 100 is presently constructed of aluminum although can be formed from a variety of metals and plastics. The material preferably exhibits some malleability and resilience to facilitate fitting and bending at the curvature of the heel portion 98 to each particular wearer. The splint 90 can also be readily molded into a plastic material or formed into a variety of other composite or metal materials. The material is further selected to provide adequate resilience to counteract torsional or rotational movement of the splint 90. The material might also exhibit properties that facilitate forming and tailor fitting such as via heating or exposure to various vibration or other energy sources.

The member 100 exhibits a generally L-shape and is formed to provide the longitudinal generally flat calf and sole portions 96 and 99 and the intermediate curved heel portion 98. The splint 90 exhibits a length at the calf portion in the range of 7 to 10 inches and a width in the range of 2½ to 3½-inches. The calf portion 96 is displaced in a range of ⅜ to ¾-inch above the heel portion 98 as noted at the spacing A-A between a tangent line to the heel portion 98 that extends parallel to the calf portion 96. The curvature of the heel portion 98 exhibits a radius of arc “R” in the range of 3 to 5 inches. The sole portion 99 extends from the heel portion 98 in the range of 7 to 10 inches. The offset displacement of the calf portion 96 relative to the heel portion 98 as noted above causes a wearer's heel to be elevated and aligns the leg and calf more parallel to the resting support 110 when the leg and boot 2 rest in a supine position and which reduces stress on the wearer's leg and knee.

The posterior surface of the member 100 is covered with a layer of closed cell foam 102. The foam 102 is adhesively bonded the full length of the member 100. A tab of hook fastener material 42 is fastened to the foam 102 at the heel portion 98 adjacent an opening 104 to the pocket space 92. A mating tab of loop fastener material 44 is attached to the end of the traction pad 70 at the opening 104 and secures the splint 90 to the traction pad 70 and boot 2 when the splint 90 is fully inserted into the pocket space 92.

An elastic or resilient tubular fabric sleeve piece 106 is mounted about the member 100 and foam 102 in the region of the curved heel portion 98. The sleeve 106 provides a slippery interface with the boot fabric 4 to prevent wear and abrasion at the heel region of the boot 2. A tab of loop fastener material 44 is secured to the end of the sleeve 106 and mates with the hook fastener tab 42 at heel portion 98 and the open end of the pocket space 92 to secure the sleeve to the splint 90.

Longitudinal slots or apertures 108 are formed into the longitudinal edges of the member 100 and support interlaced straps 94 constructed of hook or loop material. Collectively, the straps 94, pocket space 92 and overlapped hook and loop fasteners 42 and 44 at the ends of the sleeve 106 and traction pad 70 securely retain the splint 90 to the boot 2.

While the invention has been described with respect to a number of preferred constructions and considered improvements or alternatives thereto, still other constructions may be suggested to those skilled in the art. It is to be appreciated that selected ones of the foregoing features can also be used singularly or be arranged in different combinations to provide a variety of improved therapeutic footwear. The foregoing description should therefore be construed to include all those embodiments within the spirit and scope of the following claims.

What is claimed is:

1. Therapeutic footwear apparatus comprising:
a) a boot comprising a plurality of fabric panels sewn to one another and organized to wrap about and cover a portion of a wearer's leg and foot and including a plurality of tabs of hook and loop fastener material directly affixed to said panels and arranged to bind the boot to a wearer's leg and foot upon overlapping the panels onto one another;
b) a foam cradle piece mounted inside said boot and having a longitudinal channel shaped to mate with and support a wearer's calf and elevate a wearer's heel in an included arcuate cutout space adjacent a wearer's heel;
c) a flat foam extension piece affixed to a posterior surface of said cradle piece and extending to protect the bottom of a wearer's heel and sole and wrap back upon itself over the distal ends and anterior surface of a wearer's toes; and
d) a generally L-shaped splint directly mounted to the sole of the boot and having a sole portion that underlies the plantar foam extension piece and a wearer's sole, a calf portion that underlies a wearer's calf and a heel portion adapted to depress into a cushioned support surface to prevent rotation of a wearer's leg, reduce leg elevation and minimize contact pressure between the boot and heel when the boot and a wearer's leg are supported in a supine posture on the cushioned support surface.

2. Footwear apparatus as set forth in claim 1 wherein said heel portion transversely projects below said calf portion in a range of approximately ⅜ to ¾-inch.

3. Footwear apparatus as set forth in claim 1 wherein said splint is mounted in a pocket defined between a fabric layer at the sole of the boot and a traction pad mounted to the sole of said boot.

4. Footwear apparatus as set forth in claim 1 including a fabric sleeve mounted over the calf portion of said splint and having a tab of hook or loop fastener material positioned to mate with a hook or loop fastener tab at a foam cover piece mounted to the heel portion of said splint to secure said fabric sleeve to said splint.

5. Footwear apparatus as set forth in claim 1 including a layer of foam mounted to the calf, heel and sole portions of said splint.

6. Footwear apparatus as set forth in claim 1 including a plurality of straps affixed to said splint and wherein hook or loop fasteners mounted to said straps fasten to mating hook and loop fasteners at said boot.

7. Therapeutic footwear apparatus adapted to cover a wearer's leg in the region of the calf, foot, heel, sole and toes comprising:
a) a boot comprising a plurality of panels sewn to one another and oriented to overlap each other and align with a wearer's foot and calf, wherein said panels comprise an external layer of a fabric material sewn to an internal layer of a fleece material, wherein a plurality of hook or loop fastener pieces are directly affixed to surfaces of said external and internal layers and arranged to overlap and adhere the panels to each other, and wherein a pocket space is longitudinally aligned to a wearer's calf and configured between said fabric and fleece layers; and
b) a foam support comprising;
i) a cradle piece having a longitudinally contoured channel adapted to mate with and support a wearer's calf, wherein said cradle piece mounts in said pocket space, wherein an anterior surface of said channel exhibits a recessed portion and a contoured, raised portion that extends above the recessed portion in the region of a wearer's heel such that the channel is shaped and oriented to mate with the posterior surface of a wearer's calf, wherein an arcuate cutout is defined through an end of said cradle piece, and wherein a wearer's heel is suspended in the arcuate cutout, and
ii) an extension piece comprising an elongated foam sheet affixed to a posterior surface of said cradle piece adjacent an apex of said arcuate cutout and mounted between said fabric and fleece layers to extend into a toe panel to only underlie plantar surfaces of a wearer's heel, sole and toes and fold back upon itself over distal ends of a wearer's toes and over an anterior surface of the wearer's toes and foot; and
c) a generally L-shaped splint mounted in a pocket defined between a fabric layer at the sole of the boot and a traction pad mounted to the sole of said boot, wherein a sole portion of the splint underlies the foam extension piece and a wearer's sole, wherein a calf portion of the splint underlies a wearer's calf, and wherein a heel portion of the splint projects from the calf portion and is adapted to depress into a cushioned support surface to prevent rotation of a wearer's leg, reduce leg elevation and minimize contact pressure between the boot and heel when the boot and a wearer's leg are supported in a supine posture on the cushioned support surface.

8. Footwear apparatus as set forth in claim 7 including a fabric sleeve mounted over the calf portion of said splint and having a tab of hook or loop fastener material positioned to mate with a hook or loop fastener tab at a foam cover piece mounted to the heel portion of said splint to secure said fabric sleeve to said splint.

9. Footwear apparatus as set forth in claim 8 including a layer of foam mounted to the calf, heel and sole portions of said splint.

10. Footwear apparatus as set forth in claim 7 including a plurality of straps affixed to said splint and wherein hook or loop fasteners mounted to said straps fasten to mating hook and loop fasteners at said boot.

11. Therapeutic footwear apparatus comprising:
a) a boot comprising a plurality of fabric panels sewn to one another and organized to wrap about and cover a portion of a wearer's leg and foot and including a plurality of tabs of hook and loop fastener material directly affixed to said panels and arranged to bind the boot to a wearer's leg and foot upon overlapping the panels onto one another;
b) a foam cradle piece mounted inside said boot and having a longitudinal channel shaped to mate with and support a wearer's calf and elevate a wearer's heel in an included arcuate cutout space adjacent a wearer's heel;
c) a flat foam extension piece affixed to a posterior surface of said cradle piece and extending to protect the bottom of a wearer's heel and sole and wrap back upon itself over the distal ends and anterior surface of a wearer's toes; and
d) a generally L-shaped splint mounted in a pocket defined between a fabric layer at the sole of the boot and a traction pad mounted to the sole of said boot, wherein a sole portion of the splint underlies the foam extension piece and a wearer's sole, wherein a plurality of straps having hook or loop fasteners are coupled to said calf portion and mounted to overlap and fasten said straps to mating hook and loop fasteners at said boot, wherein a fabric sleeve mounts over the calf portion of said splint and includes a tab of hook or loop fastener material positioned to mate with a hook or loop fastener tab at a foam cover piece mounted to said splint to secure said fabric sleeve to said splint, wherein a calf portion of the splint underlies a wearer's calf, and wherein a heel portion of the splint projects from the calf portion and is adapted to depress into a cushioned support surface to prevent rotation of a wearer's leg, reduce leg elevation and minimize contact pressure between the boot and heel when the boot and a wearer's leg are supported in a supine posture on the cushioned support surface.

12. Footwear apparatus as set forth in claim 11 including a layer of foam mounted to the calf, heel and sole portions of said splint.
